(19) United States
(12) Patent Application Publication
(10) Pub. No.: US 2009/0131651 A1
(43) Pub. Date: May 21, 2009

(54) Synthesis of 2-substituted adenosines

(76) Inventors: Giles Albert Brown, Cambridge (GB); Edward Daniel Savory, Cambridge (GB); Jacqueline Valerie Anne Ouzman, Cambridge (GB); Alison Margaret Stoddart, Cambridge (GB)

Correspondence Address: FISH & RICHARDSON PC, P.O. BOX 1022, MINNEAPOLIS, MN, 55440-1022, US

(21) Appl. No.: 10/581,545

(22) PCT Filed: Dec. 3, 2004
(86) PCT No.: PCT/GB04/05090

§ 371 Date: Nov. 14, 2008

(30) Foreign Application Priority Data
Dec. 5, 2003 (GB) 0328319.9
Dec. 5, 2003 (GB) 0328321.5

(51) Int. Cl. C07H 19/167

(52) U.S. Cl. 536/27.61; 536/27.6; 536/27.7

(57) ABSTRACT

Synthesis of 2-substituted adenosines of formula (I) using 2-nitro pentabenzoyl adenosine, or 2-nitro pentaacetyl adenosine, as intermediate is described: Formula (I) wherein R=C₁₋₆ alkoxy (straight or branched), a phenoxy group (unsubstituted, or mono-, or di-substituted by halo, amino, CF₃—, cyano, nitro, C₁₋₄ alkyl, or C₁₋₄ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy). The methods provide improved yield and purity of product.

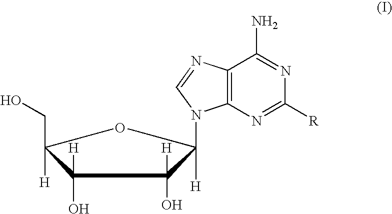

This invention relates to synthesis of 2-substituted adenosines, such as spongosine (2-methoxyadenosine) and to synthesis of intermediates for use in the synthesis of such compounds.

The natural product spongosine was first isolated from a sponge, Cryptotethia crypta, collected off the Florida coast in 1945 (Bergmann and Feeney, J. Org. Chem. 1951, 16, 981; Ibid 1956, 21, 226). Spongosine was considered an unusual nucleoside in that it was not only the first methoxypurine to be found in nature but also one of the first O-methyl compounds to be isolated from animal tissues.

Several syntheses of spongosine have been previously reported. One of the first of these to be published was by Bergmann and Stempien (J. Org. Chem. 1957, 22, 1575) in which spongosine was formed via coupling of chloromercuric 2-methoxyadenosine to 2,3,5-tri-O-benzoyl-D-ribofuranosyl chloride. This simple coupling reaction provided a crude yield of spongosine of 31% which was then recrystallised from hot water to provide spongosine which exhibited a melting point of 191-191.5° C. and an optical rotation of −43.5° (NaOH).

A variation on this theme was employed by Ojha et al. (Nucleosides and Nucleotides, 1995, 14, 1889) who initially coupled 2-ethylthioadenine with a suitably protected ribose. Subsequent adjustments of the protecting groups and oxidation gave a substrate which was reacted with sodium methoxide to yield spongosine in a yield of 87% for the final step. The purity of the target spongosine after column chromatography clean up, was proved by both elemental analysis and melting point (189-190° C.).

One of the most common methods of preparation of spongosine is via displacement of a 2-substituted chlorine atom by methoxide:

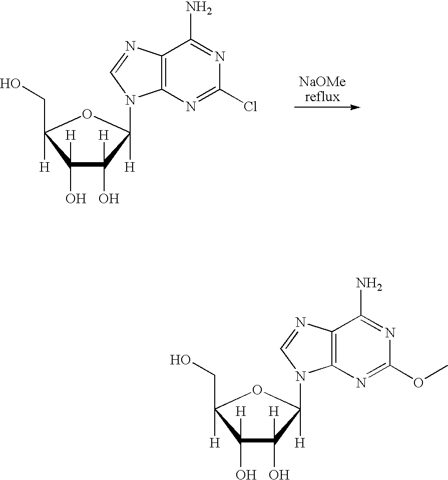

This methodology has been successfully applied by a number of groups to provide spongosine in varying yields and purity: Schaeffer et al.; J. Am. Chem. Soc. 1958, 80, 3738 (35% yield, mpt. 190-192° C.); Bartlett et al.; J. Med. Chem. 1981, 24, 947 (yield and purity not quoted), Sato et al.; Synth. Proceed. Nucleic Acid Chem. 1968, 1, 264. However, this method suffers from the disadvantage that the 2-chloroadenosine staring material is difficult to synthesise and expensive.

Spongosine was reported by Cook et al. (J. Org. Chem. 1980, 45, 4020) as a by-product in the methylation reaction of isoguanosine by methyl iodide. Both the desired 1-methylisoguanosine and the spongosine were obtained in poor crude yields (19 and 30% respectively). The crude spongosine fragment was first purified by column chromatography on silica gel (eluent: chloroform/methanol) and then recrystallised from water to provide a sample which melted between 189-192° C. (7% yield pure).

Deghati et al. (Tetrahedron Letters 41 (2000) 1291-1295) and Wanner et al. (Bioorganic & Medicinal Chemistry Letters 10 (2000) 2141-2144) describe formation of spongosine as a significant by-product in the synthesis of 2-nitroadenosine by treatment of 2-nitroadenosine pentaacetate with potassium cyanide in methanol. The 2-nitroadenosine was obtained in only 10% yield, and spongosine in 47% yield (Deghati et al.). The 2-nitroadenosine pentaacetate was produced by nitration of adenosine pentaacetate with tetrabutylammonium nitrate/trifluoroacetic anhydride (TBAN/TFAA), and (in Wanner et al.) the adenosine pentaacetate was formed by treatment of adenosine with acetic anhydride and DMAP:

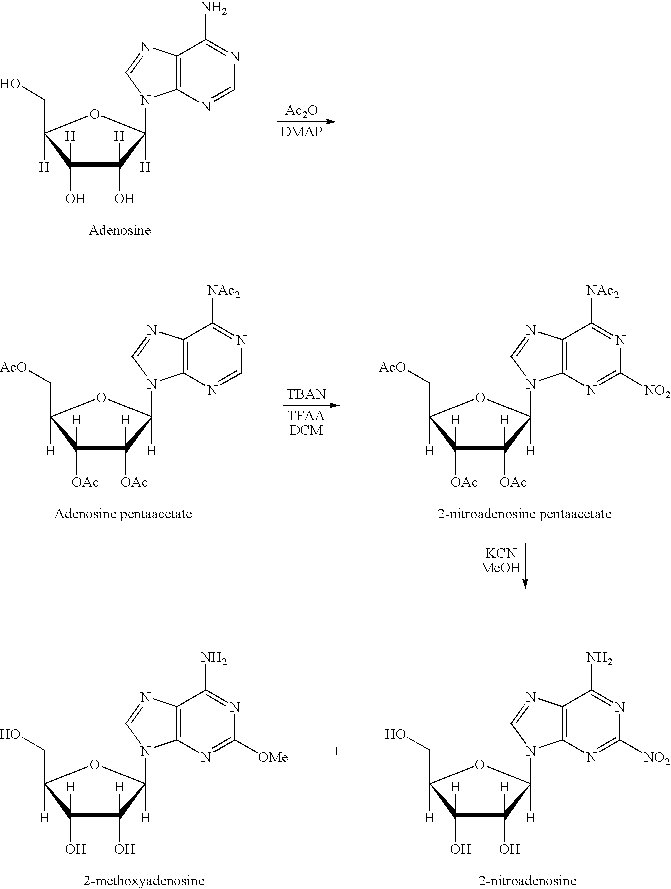

Synthesis of Spongosine (2-methoxyadenosine) According to Wanner et al.

A disadvantage of this method is that the spongosine is not produced in high yield or purity. A further disadvantage of the method is that it involves use of the toxic reagent potassium cyanide. It is desired, therefore, to provide alternative methods of synthesis of spongosine, and to improve the yield and purity of the spongosine produced.

We have appreciated that the yield and purity of spongosine produced by the method of Deghati et al., and Wanner et al. is limited by a number of factors:

i) The 2-nitroadenosine pentaacetate is contaminated with TBAN. This interferes with the subsequent methoxylation and deprotection of the 2-nitroadenosine pentaacetate (this is also the case if tetramethylammonium nitrate (The is used instead of TBAN), and adversely affects the purity and yield of the spongosine product. This is particularly problematic because TBAN is amphiphilic, and so could not be removed by aqueous work-up. In addition, because of the partial solubility of 2-nitroadenosine pentaacetate in the aqueous layer, some of this may have been lost by aqueous work-up.
ii) The adenosine pentaacetate intermediate is produced only in low yield and purity. We found that the tetra-acetylated precursor is present as a major by-product.
iii) The fifth acetate group of the penta-acetyl compounds is labile, and this results in decomposition of these compounds to tetra-acetyl compounds. For example, we purified adenosine pentaacetate by column chromatography, but there was evidence to suggest that the compound decomposed during this process. Attempts to recrystallise this compound were not successful and it was amorphous rather than crystalline in nature.

We have found, surprisingly, that the purity and yield of spongosine and other 2-substituted adenosines may be greatly improved by use of benzoyl protecting groups.

According to the invention there is provided a method of synthesis of a 2-substituted adenosine of formula I, which comprises converting 2-nitro pentabenzoyl adenosine to the 2-substituted adenosine:

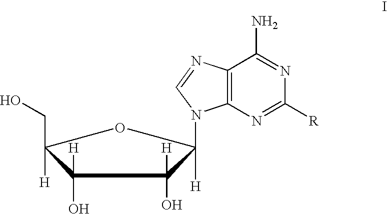

wherein R=C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy).

Preferably R=methoxy, ethoxy, propoxy, butoxy, pentyloxy, hexyloxy, phenoxy, benzyloxy, or benzoyl.

We have found that 2-nitro-pentabenzoyl adenosine has increased organic solubility, stability and crystallinity compared to 2-nitroadenosine pentaacetate. The 2-nitro-pentabenzoyl adenosine is, therefore easier to handle than 2-nitroadenosine pentaacetate, and can be made in higher yield and purity than this compound. The yield and purity of the spongosine produced is thereby also improved. Other 2-substituted adenosines can also be produced in high yield and purity using 2-nitro-pentabenzoyl adenosine as intermediate.

Preferably the 2-nitro-pentabenzoyl adenosine is converted to the 2-substituted adenosine by reacting the 2-nitro-pentabenzoyl adenosine with a suitable anion (for example C₁₋₆ alkoxide anion, or a phenoxide anion), or by deprotecting the 2-nitro-pentabenzoyl adenosine and reaction with a suitable anion (for example C₁₋₆ alkoxide anion, or a phenoxide anion) To synthesise spongosine this may be achieved by reaction with potassium cyanide and methanol as detailed in Deghati et al., and Wanner et al. However, it is preferred that less toxic sources of the methoxide anion are used. Preferred sources are MeOH/NaOMe, MeOH/n-BuLi, MeOH/NaOH, MeOH/NaH, or MeOH/KO^{t}Bu.

A preferred method of methoxylating 2-nitro-pentabenzoyl adenosine is described in Example 4 below.

Other 2-substituted adenosines of formula I may be made by treatment of 2-nitro-pentabenzoyl adenosine with sodium hydroxide, sodium hydride, butyl lithium, or KO^{t}Bu, and an appropriate alcohol (for example C₁₋₆ alcohol, or phenol). KO^{t}Bu may be used with phenol.

2-nitro pentabenzoyl adenosine is also provided according to the invention.

There is further provided according to the invention use of 2-nitro pentabenzoyl adenosine in the synthesis of a 2-substituted adenosine of formula I.

Preferably methods of the invention further comprise converting pentabenzoyl adenosine to 2-nitro-pentabenzoyl adenosine.

According to a her aspect of the invention, there is provided a method of synthesising 2-nitro-pentabenzoyl adenosine or a 2-substituted adenosine of formula I, which comprises converting pentabenzoyl adenosine to 2-nitro-pentabenzoyl adenosine.

Conversion of pentabenzoyl adenosine to 2-nitro-pentabenzoyl adenosine may be achieved by nitrating pentabenzoyl adenosine with a suitable nitrating reagent, such as tetrabutylammonium nitrate (TBAN) or tetramethylammonium nitrate (TMAN). Preferably nitration is carried out using TBAN or TMAN with trifluoroacetic anhydride (TBAN/TFAA, or TMAN/TFAA). Preferably the TBAN/TFAA or TMAN/TFAA is in dichloromethane (DCM).

2-nitro-pentabenzoyl adenosine has increased organic solubility and crystallinity compared to 2-nitroadenosine pentaacetate. A particular advantage of these properties is that, in contrast to 2-nitroadenosine pentaacetate, much or all of the TBAN or TMAN can be removed from the 2-nitro-pentabenzoyl adenosine by aqueous work-up, preferably followed by recrystallisation. It may be preferred that TMAN is used as nitrating agent rather than TBAN, since we have found that TMAN is easier to remove than TBAN. Preferably 3-5 washes are carried out in the aqueous work-up, and preferably 2 or 3 recrystallisations are carried out.

For example, aqueous work-up of the 2-nitro-pentabenzoyl adenosine produced may be carried out by dissolving the compound in an organic solvent (such as ethyl acetate or DCM), and washing the resulting solution with water. In general, a minimum of three washes has been found to be required to remove a large proportion of the TBAN or TMAN. However, five washes are generally carried out to ensure as much TBAN or TMAN as possible is removed.

Recrystallisation may be carried out by removing the organic solvent after the solution has been washed with water, dissolving the 2-nitro-pentabenzoyl adenosine in EtOAc/ethanol, or dichloromethane/ethanol, and crystallising the 2-nitro-pentabenzoyl adenosine from this solution.

We have found that the crude product of the nitration reaction with TBAN/TFAA could not be recrystallised because of the large amount of TBAN present. However, after aqueous work-up the compound could be readily recrystallised from a mixture of EtOAc or CH₂Cl₂ and ethanol. Impurities other than TBAN were also present in the mixture after the work-up process and these could be removed by recrystallisation. A minimum of one recrystallisation may be sufficient but sometimes two or three recrystallisations may be required for satisfactory removal of these impurities.

The increased organic solubility of the penta-benzoyl compounds compared with the penta-acetyl compounds ensures that only an insignificant amount of compound is lost by aqueous work-up and recrystallisation.

Preferred methods of nitrating pentabenzoyl adenosine are described in Examples 2 and 3 below.

Preferably methods of the invention further comprise converting adenosine to pentabenzoyl adenosine.

According to the invention there is further provided a method of synthesising pentabenzoyl adenosine, 2-nitro-pentabenzoyl adenosine, or a 2-substituted adenosine of formula I, which comprises converting adenosine to pentabenzoyl adenosine.

Conversion of adenosine to pentabenzoyl adenosine may be achieved by benzoylating adenosine with a suitable benzoylating reagent, such as benzoyl chloride. A suitable base, such as pyridine, should also be used. Dimethylformamide (DMF) may be used as solvent, but preferably the adenosine is dissolved/suspended in pyridine as this gives cleaner results.

A preferred method of benzoylating, adenosine is described in Example 1 below.

An advantage of use of pentabenzoyl adenosine is that it can be more readily purified than adenosine pentaacetate. For example, pentabenzoyl adenosine was purified by aqueous work-up followed by recrystallisation. This was preferable to purification of adenosine pentaacetate which involved column chromatography during which some decomposition and loss of product occurred.

There is also provided according to the invention use of pentabenzoyl adenosine in the synthesis of 2-nitro pentabenzoyl adenosine, or a 2-substituted adenosine of formula I.

There is further provided according to the invention use of a benzoylating reagent in the synthesis of a 2-substituted adenosine of formula I.

There is also provided according to the invention a 2-substituted adenosine, 2-nitro-pentabenzoyl adenosine, or pentabenzoyl adenosine synthesised by a method of the invention.

Methods of the invention allow synthesis of products more easily, and with greater yield and purity than the known method of Deghati et al. and Wanner et al. which uses acetyl protecting groups. We have appreciated that this is due to the increased organic solubility, stability and crystallinity of the compounds used in the invention.

According to an alternative aspect of the invention there is provided a method of synthesising a 2-substituted adenosine of formula I, which comprises: nitrating adenosine pentaacetate using TBAN or TMAN to produce 2-nitroadenosine pentaacetate; reducing the amount of TBAN or TMAN contaminating the 2-nitroadenosine pentaacetate; and then producing the 2-substituted adenosine from the 2-nitroadenosine pentaacetate:

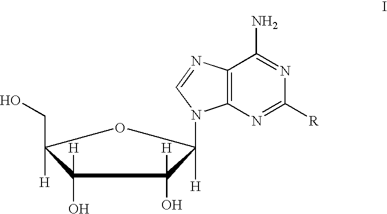

wherein R=C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃— cyano, nitro C₁₋₆ alkyl, or C₁₋₆ alkoxy).

Preferably R is methoxy, ethoxy, propoxy, butoxy, pentyloxy, hexyloxy, phenoxy, benzyloxy, or benzoyl.

It has surprisingly been found that an effective method of reducing the amount of TBAN and TMAN contaminant is trituration of the 2-nitroadenosine pentaacetate with isopropanol, followed by washing with water. This can significantly improve the purity and yield of the spongosine or other 2-substituted adenosine product.

There is also provided according to the invention a method of reducing the amount of TBAN or TMAN contaminating 2-nitroadenosine pentaacetate formed by nitration of adenosine pentaacetate with TBAN or TAN, which comprises triturating the 2-nitroadenosine pentaacetate with isopropanol and washing the triturated 2-nitroadenosine pentaacetate with water to reduce the amount of TBAN or TMAN.

There is further provided according to the invention 2-nitroadenosine pentaacetate produced by such methods.

Preferably nitration is carried out using TBAN or TMAN with trifluoroacetic anhydride (TBAN/TFAA, or TMAN/TFAA). Preferably the TBAN/TFAA or TMAN/TFAA is in dichloromethane (DCM). A preferred method of nitration of adenosine pentaacetate is described in example 5 below.

2-nitroadenosine pentaacetate may be converted to the 2-substituted adenosine by deprotecting the 2-nitroadenosine pentaacetate and reaction with a suitable anion (for example a C₁₋₆ alkoxide anion, or a phenoxide anion). To synthesise spongosine this may be achieved by reaction with potassium cyanide and methanol as detailed in Deghati et al., and Wanner et al. However, it is preferred that less toxic sources of the methoxide anion are used. Preferred sources are MeOH/NaOMe, MeOH/n-BuLi, MeOH/NaOH, MeOH/NaH, or MeOH/KO^{t}Bu. A preferred method of conversion of 2-nitroadenosine pentaacetate to spongosine is described in example 5 below. It is believed that other 2-substituted adenosines may be synthesised by treatment of the 2-nitroadenosine pentaacetate with an appropriate C₂₋₆ alcohol, or a phenol, and sodium hydroxide.

According to a further aspect of the invention there is provided a method of synthesising spongosine which comprises treating 2-nitroadenosine pentaacetate with MeOH/NaOMe, MeOH/n-BuLi, MeOH/NaOH or MeOH/NaH to form spongosine.

There is also providing according to the invention a method of synthesising a 2-substituted adenosine of formula I, excluding spongosine, which comprises deprotecting 2-nitroadenosine pentaacetate, and reaction with a C₂₋₆ alkoxide anion, or a phenoxide anion. It is believed that this may be achieved by reaction with an appropriate C₂₋₄ alcohol, or a phenol, and sodium hydroxide (or NaH, BuLi, or KO^{t}Bu).

Methods of the invention may further comprise converting adenosine to adenosine pentaacetate. This may be achieved by the method detailed by Deghati et al., and Wanner et al. However, we have appreciated that adenosine pentaacetate is produced only in low yield and purity using this method, and that the tetra-acetylated precursor is present as a major by-product.

We have found that the yield and purity of the 2-substituted adenosine product may be improved if methods of the invention further comprise acylating adenosine to form an O-tri-acetyl and/or tetra-acetyl derivative of adenosine, isolating the derivative(s), and acylating the isolated derivative(s) to produce the adenosine pentaacetate intermediate.

According to a further aspect of the invention there is provided a method of synthesising adenosine pentaacetate, 2-nitroadenosine pentaacetate, or a 2-substituted adenosine of formula I, which includes the following steps: acylating adenosine to form an O-tri-acetyl and/or tetra-acetyl derivative of adenosine, isolating the derivative(s), and acylating the isolated derivative(s) to produce adenosine pentaacetate.

The O-tri-acetyl and/or tetra-acetyl derivative can be isolated using column chromatography.

The adenosine may then be nitrated to form 2-nitroadenosine pentaacetate. The 2-nitroadenosine pentaacetate may then be converted to a 2-substituted adenosine of formula I, for example using a method of the invention.

We have also found that the fifth acetate group of the penta-acetyl compounds is labile, and this results in decomposition of these compounds to tetra-acetyl compounds. For example, we purified adenosine pentaacetate by column chromatography, but there was evidence to suggest that the compound decomposed during his process. Attempts to recrystallise this compound were not successful and it was amorphous rather than crystalline in nature.

We have appreciated that the yield and purity of the 2-substituted adenosine product may be improved if methods of the invention alternatively or additionally further comprise washing the adenosine pentaacetate intermediate to reduce the amount of contaminating adenosine tetraacetate before nitrating the washed adenosine pentaacetate.

According to a further aspect of the invention there is provided a method of synthesising adenosine pentaacetate, 2-nitroadenosine pentaacetate, or a 2-substituted adenosine of formula I, which includes the following steps: acylating adenosine or an acylated derivative of adenosine to form adenosine pentaacetate; and washing the adenosine pentaacetate to reduce the amount of contaminating adenosine tetraacetate.

To wash the adenosine pentaacetate, it is preferably dissolved in chloroform and washed with acetic acid solution (preferably 1M).

The adenosine pentaacetate may then be nitrated to form 2-nitroadenosine pentaacetate. The 2-nitroadenosine pentaacetate may then be converted to a 2-substituted adenosine of formula I, for example using a method of the invention.

It is thought that 2-nitroadenosine pentaacetate may be toxic. Thus, it may be desirable to ensure that a 2-substituted adenosine produced from 2-nitroadenosine pentaacetate is contaminated with as little 2-nitroadenosine pentaacetate as possible. According to the invention this may be achieved by converting the 2-nitroadenosine pentaacetate to 2-chloroadenosine pentaacetate before converting the 2-chloroadenosine pentaacetate to the 2-substituted adenosine.

It is believed that conversion of 2-nitroadenosine pentaacetate to 2-chloroadenosine pentaacetate may be achieved by chlorinating the 2-nitroadenosine pentaacetate with a suitable chlorinating reagent, such as ammonium chloride.

According to a further aspect of the invention there is provided a method of synthesis of a 2-substituted adenosine of formula I which comprises converting 2-chloroadenosine pentaacetate to the 2-substituted adenosine.

There is also provided according to the invention use of penta-acetylated 2-chloroadenosine in the synthesis of a 2-substituted adenosine.

It is believed that 2-chloroadenosine pentaacetate may be converted to the 2-substituted adenosine by deprotecting the 2-chloroadenosine pentaacetate and reaction with a suitable anion (for example a C₁₋₆ alkoxide anion, or a phenoxide anion). To synthesise spongosine it is believed that this may be achieved by reaction with potassium cyanide and methanol as detailed in Deghati et al., and Wanner et al. However, it is preferred that less toxic sources of the methoxide anion are used. Preferred sources are MeOH/NaOMe, MeOH/n-BuLi, MeOH/NaOH, or MeOH/NaH. It is believed that other 2-substituted adenosines may be synthesised using an appropriate C₂₋₁₆ alcohol, or a phenol, and sodium hydroxide (or BuLi, NaH, or KOtBu).

There is also provided according to the invention a 2-substituted adenosine of formula I, or an intermediate for use in synthesis of a 2-substituted adenosine of formula I, produced by a method of the invention.

Methods of the invention can be used to synthesise 2-substituted adenosines in high yield and purity. For example, we have been able to synthesise spongosine which is >96% pure.

Embodiments of the invention are now described by way of example only with reference to the accompanying Schemes 1 and 2 which show preferred methods of synthesis of 2-methoxyadenosine (spongosine).

### EXAMPLE 1

### Preparation of Pentabenzoyl Adenosine

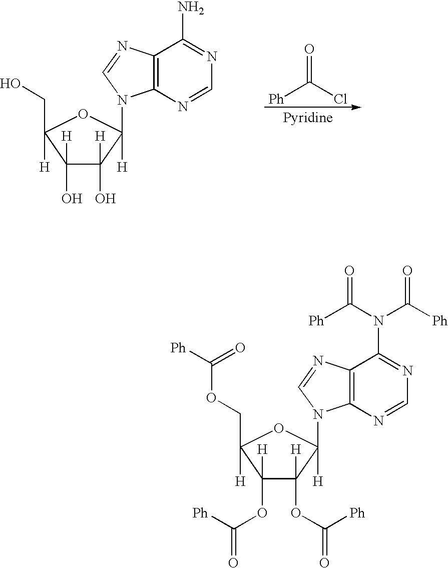

To a suspension/solution of adenosine (2.00 g, 7.47 mmol) in pyridine (20 cm³) add benzoyl chloride (7.35 g, 6.07 cm³, 52.29 mmol). Heat at 65° C. for 41 h, pour reaction mixture onto ethanol (20 cm³). Solvent removed in vacuo. Residue partitioned between DCM (300 cm³), washed with water (100 cm³), aqueous layer washed with DCM (3×50 cm³), organic layers combined and washed with water (2×100 cm³), brine (100 cm³), dried (MgSO₄). Solvent removed in vacuo, residue purified by recrystallisation from acetone/EtOH to give the desire product (5.660 g, 96.2%) as a colourless solid. LCMS: 788 (M+H).

### EXAMPLE 2

### Preparation of 2-Nitro-Pentabenzoyl Adenosine Using TMAN/TFAA as Nitrating Reagent

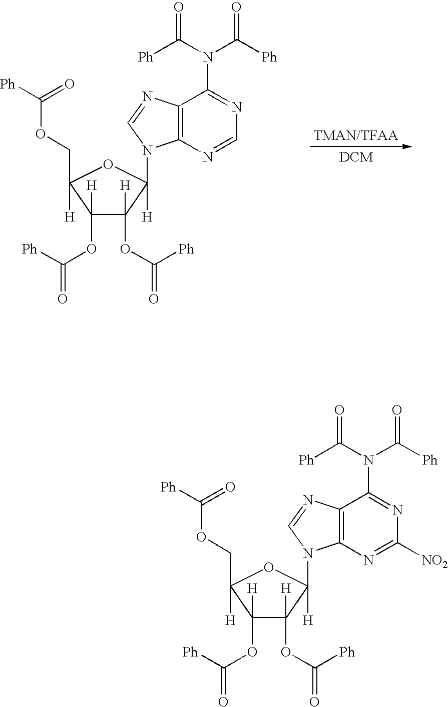

To a suspension of tetramethylammonium nitrate (137 g, 11.4 mmol) in DCM (40 cm³) charge trifluoroacetic anhydride (2.40 g, 1.62 cm³, 114 mmol). Stir at room temperature for 1.5 h, cool to 0° C. and add a solution of pentabenzoyl adenosine (6.00 g, 7.62 mmol) in DCM (50 cm³). Allow to warm to room temperature over 14 h, solvent removed in vacuo [Temperature of rotary evaporator water bath is kept at 30° C. or below]. Residue dissolved in EtOAc (200 cm³), washed with water (3×150 cm³), brine (50 cm³), dried (MgSO₄). Solvent removed in vacuo, residue purified by recrystallisation from DCM/EtOH (twice) to give the desire product (5.59 g, 88.2%) as an off white solid. ¹H NMR (400 MHz, CDCl₃): 4.79 (1H, dd, J=11.5, 4.2 Hz), 4.92 (2H, m), 6.08 (1H, t, J=5.6 Hz), 6.16 (1H, dd, J=5.8, 4.4 Hz), 6.57 (1H, d, J=5.4 Hz), 7.39 (10H, m), 7.55 (5H, m), 7.85 (4H, m), 7.92 (2H, m), 8.04 (4H, m) and 8.44 (1H, s). LCMS: 833 M+H) and 855 (M+Na).

### EXAMPLE 3

### Preparation of 2-Nitro-Pentabenzoyl Adenosine Using TBAN/TFAA as Nitrating Reagent

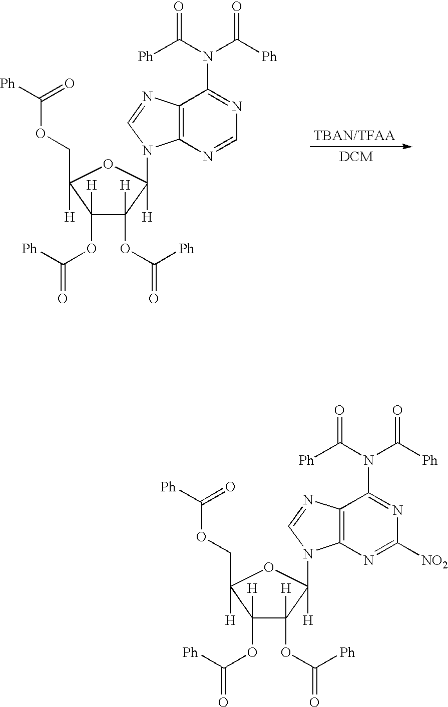

To a solution of tetrabutylammonium nitrate (1.16 g, 3.81 mmol) in DCM (20 cm³) charge trifluoroacetic anhydride (0.80 g, 0.538 cm³ 3.81 mmol). Stir at 0° C. for 0.5 h, then add a solution of pentabenzoyl adenosine (2.00 g, 2.54 mmol) in DCM (20 cm³) at 0° C. (optionally cover reaction vessel in silver foil). Allow to warm to room temperature over 14 h, reaction mixture poured onto ice/water, separate aqueous layer and extract with DCM (40 cm³), organic layers combined, solvent removed in vacuo [Temperature of rotary evaporator water bath is kept at 30° C. or below]. Residue dissolved in EtOAc (150 cm³), washed with water (5×75 cm³), brine (50 cm³), dried (MgSO₄). Solvent removed in vacuo, residue purified by recrystallisation from DCM/EtOH (twice) to give the desired product (1.604 g, 75.9%) as a pale yellow solid. ¹H NMR (400 MHz, CDCl₃): 4.79 (1H, dd, J=1.1.5, 4.2 Hz), 4.92 (2H, m), 6.08 (1H, t, J=5.6 Hz), 6.16 (1H, dd, J=5.8, 4.4 Hz), 6.57 (1H, d, J=5.4 Hz), 7.39 (10H, m), 7.55 (5H, m), 7.85 (4H, m), 7.92 (2H, m), 8.04 (4H, m) and 8.44 (1H, s). LCMS: 833 (M+H) and 855 (M+Na).

### EXAMPLE 4

### Preparation of 2-Methoxy Adenosine (Spongosine)

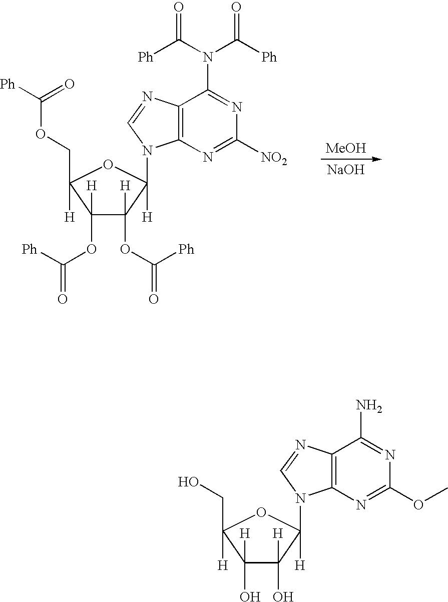

To a suspension of 2-nitro-pentabenzoyl adenosine (0.52 g, 0.62 mmol) in MeOH (10 cm³) charge a solution of NaOH (0.15 g, 3.70 mmol) in MeOH (10 cm³). Stir at room temperature for 16 h, a red solution is obtained. Solvent removed in vacuo, residue dissolved in water and neutralised with 0.2M HCl (dropwise so as to prevent over acidification and resulting depurination). Solvent removed in vacuo, residue dissolved in MeOH:Water (1:1) (approx. 40 cm³) [requires heating], reaction mixture placed in freezer overnight (−20° C.). Desired product precipitates out of reaction mixture, filtration gives the title compound (0.100 g, 54%) as a pale yellow solid. LCMS: 298 (M+H), small impurity 329 (M+H). Further purification can be carried out using reverse phase chromatography. ¹H NMR (400 MHz, CDCl₃): 3.52 (1H, m), 3.60 (1H, m), 3.78 (3H, s), 3.89 (1H, dd, J=7.2, 3.9 Hz), 4.12 (1H, m), 4.56 (1H, dd, J=11.3, 6.1 Hz), 5.10 (1H, m), 5.11 (1H, d, J=4.7 Hz), 5.35 (1H, d, J=6.2 Hz), 5.75 (1H, d, J=6.2 Hz), 7.27 (2H, br, s) and 8.11 (1H, s).

### EXAMPLE 5

### Preparation of Adenosine Pentaacetate

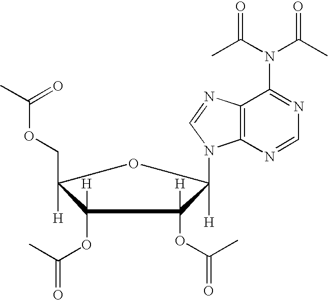

To a solution of adenosine (1.0 g, 3.74 mmol) in acetic anhydride (10 mL) was added sodium hydride (60% in mineral oil, 0.9 g, 22.5 mmol) and the mixture was heated at 110° C. for 20 h. Reaction mixture was allowed to cool to room temperature, then poured onto ice/NaHCO₃ (250 mL). EtOAc (150 mL) was added and organic phase washed with water (3×100 cm³), dried (MgSO₄) and the solvent removed in vacuo. The crude product was purified by silica gel chromatography (silica gel 60), eluting with EtOAc:Heptane (1:1), increasing to EtOAc to give the desired product (0.6 g, 31%).

### Preparation of 2-Nitro-Adenosine Pentaacetate

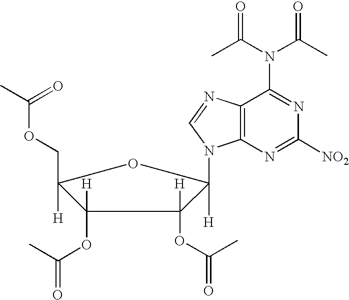

To a suspension of tetramethylammonium nitrate (642 mg, 4.72 mmol) in DCM (10 ml) was added trifluoroacetic anhydride (0.68 mL, 4.72 mmol) and the resulting suspension stirred at room temperature for 1 h before cooling to 0° C. A solution of adenosine pentaacetate (1.50 g, 3.14 mmol) in DCM (10 mL) was added and the solution was allowed to warm to room temperature over 2.5 h. The crude product was then washed with brine, dried (MgSO₄) and the solvent was removed in vacuo to give the target product as a pale brown solid foam (1.366 g, 83%).

### Preparation of 2-Methoxy Adenosine (Spongosine)

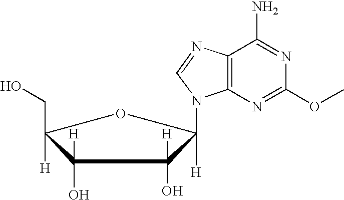

To a solution of 2-nitro-adenosine pentaacetate (275 mg. 0.53 mmol) (in MeOH) at room temperature was added NaOMe (71 mg, 1.3 mmol) and the mixture stirred for 3 h. Ammonium chloride (70 mg, 1.3 mmol) was added and the reaction mire concentrated in vacuo to give a yellow oil. The crude product was purified by silica gel chromatography, eluting with EtOAc, increasing to EtOAc:MeOH (15:1) and then recrystallisation from isopropanol to give the target product as a white solid (70 mg, 47%).

Instead of ammonium chloride, citric acid solution or 0.2 HCL could preferably be used.

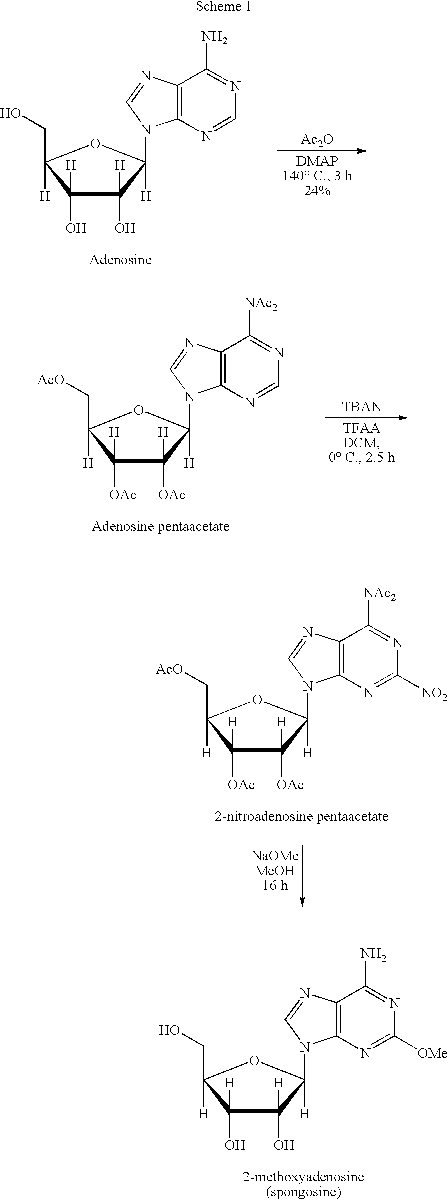

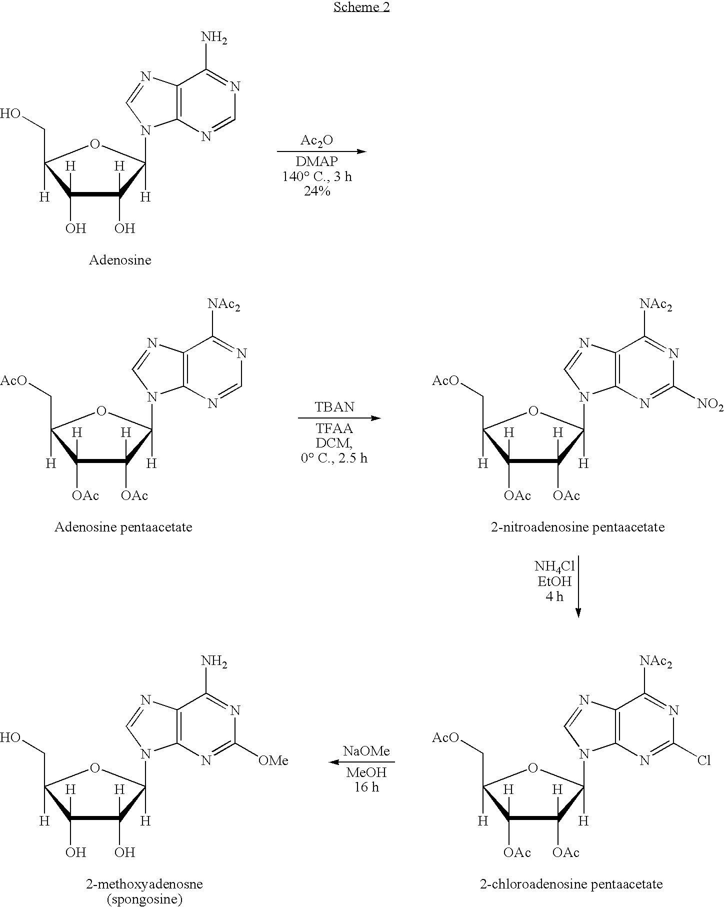

1. A method of synthesising a 2-substituted adenosine of formula I, which comprises converting 2-nitro-pentabenzoyl adenosine to the 2-substituted adenosine: 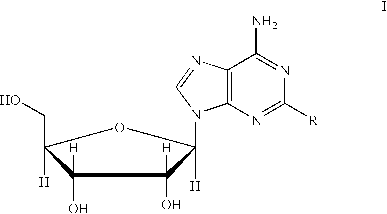
wherein R=C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy).

2. A method according to claim 1, wherein R=methoxy, ethoxy, propoxy, butoxy, pentyloxy, hexyloxy, phenoxy, benzyloxy, or benzoyl.

3. A method according to claim 1, wherein 2-nitro-pentabenzoyl adenosine is converted to the 2-substituted adenosine by deprotection, and reaction with C₁₋₆ alkoxide anion, or a phenoxide anion.

4. A method according to claim 3, wherein the anion is methoxide anion produced from MeOH/NaOMe, MeOH/n-BuLi, MeOH/NaOH, MeOH/NaH, or MeOH/KO^{t}Bu.

5. A method according to claim 1, which further comprises converting pentabenzoyl adenosine to 2-nitro-pentabenzoyl adenosine.

6. A method of synthesising 2-nitro-pentabenzoyl adenosine which comprises converting pentabenzoyl adenosine to 2-nitro-pentabenzoyl adenosine.

7. A method according to claim 5, wherein pentabenzoyl adenosine is converted to 2-nitro-pentabenzoyl adenosine by nitrating pentabenzoyl adenosine using tetrabutylammonium nitrate (TBAN), or tetramethylammonium nitrate (TMAN) as nitrating reagent.

8. A method according to claim 7, which further comprises reducing the amount of TBAN or TMAN contaminating the 2-nitro-pentabenzoyl adenosine after the nitration reaction.

9. A method according to claim 8, wherein the amount of TBAN or TMAN is reduced by washing the 2-nitro-pentabenzoyl adenosine with water.

10. A method according to claim 9, which further comprises recrystallising the 2-nitro-pentabenzoyl adenosine after washing with water.

11. A method according to claim 5, which further comprises converting adenosine to pentabenzoyl adenosine.

12. A method of synthesising pentabenzoyl adenosine or 2-nitro-pentabenzoyl adenosine which comprises converting adenosine to pentabenzoyl adenosine.

13. A method according claim 11, wherein adenosine is benzoylated using benzoyl chloride.

14. 2-nitro pentabenzoyl adenosine.

15. Use of 2-nitro pentabenzoyl adenosine in the synthesis of a 2-substituted adenosine of formula I.

16. Use of pentabenzoyl adenosine in the synthesis of 2-nitro-pentabenzoyl adenosine, or a 2-substituted adenosine of formula I.

17. Use of a benzoylating reagent in the synthesis of a 2-substituted adenosine of formula I.

18. A method of reducing the amount of TBAN or TMAN contaminating 2-nitro-pentabenzoyl adenosine formed by nitration of pentabenzoyl adenosine with TBAN or TMAN, which comprises washing the 2-nitro-pentabenzoyl adenosine with water.

19. A method according to claim 18 which further comprises recrystallising the 2-nitro-pentabenzoyl adenosine after washing with water.

20. A method of synthesising a 2-substituted adenosine of formula I, which comprises: nitrating adenosine pentaacetate using tetrabutylammonium nitrate (TBAN) or tetramethylammonium nitrate (TMAN) to produce 2-nitroadenosine pentaacetate; reducing the amount of TBAN or TMAN contaminating the 2-nitroadenosine pentaacetate; and then producing the 2-substituted adenosine from the 2-nitroadenosine pentaacetate: 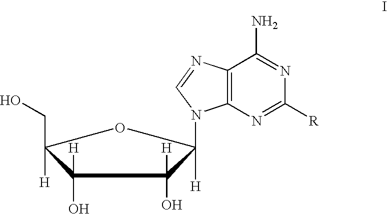
wherein R=C₁₋₆ alkoxy (straight or branched), a phenoxy group (unsubstituted, or mono-, or di-substituted by halo, amino, CF₃—, cyano, nitro, C₁₋₆ alkyl, or C₁₋₆ alkoxy), a benzyloxy group (unsubstituted, or mono-, or di-substituted by halo, amino, CF₃—, cyano, nitro, C₁₋₆ alkyl, or C₁₋₆ alkoxy), or a benzoyl group (unsubstituted, or mono-, or di-substituted by halo, amino, CF₃—, cyano, nitro, C₁₋₆ alkyl, or C₁₋₆ alkoxy).

21. A method according to claim 20, wherein the amount of TBAN or TMAN contaminant is reduced by triturating the 2-nitroadenosine pentaacetate with isopropanol and washing the triturated 2-nitroadenosine pentaacetate with water.

22. A method according to claim 20, wherein the 2-substituted adenosine is produced from the 2-nitroadenosine pentaacetate by deprotecting the 2-nitroadenosine pentaacetate and reaction with a C₁₋₆ alkoxide anion or a phenoxide anion.

23. A method according to claim 20, wherein the 2-substituted adenosine is 2-methoxy adenosine, and this is produced from the 2-nitroadenosine pentaacetate by reaction with methoxide anion from methanol/NaOMe, methanol/n-BuLi, methanol/NaOH, methanol/NaH, or methanol/KO^{t}Bu.

24. A method according to claim 20, which further comprises synthesising the adenosine pentaacetate by acylating adenosine.

25. A method according to claim 24, wherein the adenosine is acylated to form an O-tri-acetyl and/or tetra-acetyl derivative of adenosine, the derivative(s) is isolated, and the isolated derivative(s) is acylated to produce adenosine pentaacetate.

26. A method according to claim 24, or 25 which further comprises washing the adenosine pentaacetate to remove contaminating adenosine tetraacetate before nitrating the washed adenosine pentaacetate to form the 2-nitroadenosine pentaacetate.

27. A method of synthesising a 2-substituted adenosine of formula I, which comprises acylating adenosine to form an O-tri-acetyl and/or tetra-acetyl derivative of adenosine, isolating the derivative(s), acylating the isolated derivative(s) to produce adenosine pentaacetate, and producing the 2-substituted adenosine from the adenosine pentaacetate.

28. A method according to claim 27 which further comprises washing the adenosine pentaacetate to reduce the amount of contaminating adenosine tetraacetate before producing the 2-substituted adenosine from the washed adenosine pentaacetate.

29. A method of synthesising a 2-substituted adenosine of formula I, which comprises acylating adenosine, or an acylated derivative of adenosine, to form adenosine pentaacetate, washing the adenosine pentaacetate to reduce the amount of contaminating adenosine tetraacetate, and producing the 2-substituted adenosine from the washed adenosine pentaacetate.

30. A method according to claim 27, which further comprises nitrating the adenosine pentaacetate to produce 2-nitroadenosine pentaacetate, and producing the 2-substituted adenosine from the 2-nitroadenosine pentaacetate.

31. A method according to claim 30, wherein the 2-substituted adenosine is 2-methoxyadenosine, and is produced by reacting methoxide anion from methanol/NaOMe, methanol/n-BuLi, methanol/NaOH, methanol/NaH, or methanol/KO^{t}Bu with the 2-nitroadenosine pentaacetate.

32. A method according to claim 20, which further comprises converting 2-nitroadenosine pentaacetate to 2-chloroadenosine pentaacetate before producing the 2-substituted adenosine from the 2-chloroadenosine pentaacetate.

33. A method of synthesising a 2-substituted adenosine, which comprises converting 2-chloroadenosine pentaacetate to the 2-substituted adenosine.

34. A method according to claim 33, which further comprises producing the 2-chloroadenosine pentaacetate from 2-nitroadenosine pentaacetate.

35. A method according to claim 32, wherein the 2-substituted adenosine is 2-methoxyadenosine, and the 2-chloroadenosine pentaacetate is converted to 2-methoxyadenosine by reaction with methoxide anion from methanol/NaOMe, methanol/n-BuLi, methanol/NaOH, or methanol/NaH with the 2-nitroadenosine pentaacetate.

36. A 2-substituted adenosine synthesised by a method according to claim 20.

37. A method of synthesising 2-methoxyadenosine, which comprises reacting methoxide anion from methanol/NaOMe, methanol/n-BuLi, methanol/NaOH, methanol/NaH, or methanol/KO^{t}Bu with 2-nitroadenosine pentaacetate.

38. A method of synthesising 2-methoxyadenosine, which comprises the steps shown in scheme 1 or 2.

39. (canceled)

40. 2-methoxyadenosine which is >96% pure.

41. A method of synthesising 2-nitroadenosine pentaacetate, which comprises nitrating adenosine pentaacetate using TBAN or TMAN to produce 2-nitroadenosine pentaacetate, and reducing the amount of TBAN or TMAN contaminating the 2-nitroadenosine pentaacetate.

42. A method according to claim 41, wherein the amount of TBAN or TMAN contaminant is reduced by triturating the 2-nitroadenosine pentaacetate with isopropanol and washing the triturated 2-nitroadenosine pentaacetate with water.

43. A method of synthesising adenosine pentaacetate, 2-nitroadenosine pentaacetate, or a 2-substituted adenosine of formula I, which includes the following steps: acylating adenosine to form an O-tri-acetyl and/or tetra-acetyl derivative of adenosine, isolating the derivative(s), and acylating the isolated derivative(s) to produce adenosine pentaacetate.

44. A method of synthesising adenosine pentaacetate, 2-nitroadenosine pentaacetate, or a 2-substituted adenosine of formula I, which includes the following steps: acylating adenosine or an acylated derivative of adenosine to form adenosine pentaacetate; and washing the adenosine pentaacetate to reduce the amount of contaminating adenosine tetraacetate.
